(19) United States
(12) Patent Application Publication
(10) Pub. No.: US 2006/0058582 A1
(43) Pub. Date: Mar. 16, 2006

(54) Disposable shapelocking system

(75) Inventors: Tracy D. Maahs, Rancho Santa Margarita, CA (US); Vahid Saadat, Saratoga, CA (US); Chris Rothe, San Jose, CA (US); Tung Thanh Le, Tustin, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: USGI Medical Inc., San Clemente, CA (US)

(21) Appl. No.: 11/238,298

(22) Filed: Sep. 28, 2005

Related U.S. Application Data
(63) Continuation-in-part of application No. 10/281,462, filed on Oct. 25, 2002, now Pat. No. 6,960,163.
Application No. 10/281,462 is a continuation-in-part of application No. 10/173,203, filed on Jun. 13, 2002.
Application No. 10/281,462 is a continuation-in-part of application No. 10/173,220, filed on Jun. 13, 2002, now Pat. No. 6,783,491.
Application No. 10/281,462 is a continuation-in-part of application No. 10/173,227, filed on Jun. 13, 2002, now Pat. No. 6,790,173.
Application No. 10/281,462 is a continuation-in-part of application No. 10/173,238, filed on Jun. 13, 2002, now Pat. No. 6,837,847.

(51) Int. Cl. A61B 1/00

(52) U.S. Cl. 600/144; 600/141

(57) ABSTRACT

Disposable shapelocking systems are disclosed herein. A shapelock assembly generally comprises an elongate body defining at least one lumen therethrough for advancement of an endoscope or other endoscopic instruments therethrough. A handle assembly can be actuated to compress nested links against one another to transition the elongate body from a flexible state to a rigid shape-locked state. One or more of the nested links can be made from a particular thermoplastic either alone or in combination with one or more reinforcing structures. Such structures can include a reinforcing ring integrated with the link on an inner, outer, or lower surface of the link. Alternatively, the link can be coated or layered to enhance its strength. Additionally, different portions of the shapelock body can be made from different types of links depending upon the loads imparted upon the various portions of the shapelock body.

### CROSS-REFERENCES TO RELATED APPLICATIONS

This is a continuation-in-part of U.S. patent application Ser. No. 10/281,462 (Attorney Docket No. 021486-002212US), filed Oct. 25, 2002, which is a continuation-in-part of U.S. patent application Ser. No. 10/173,203 (Attorney Docket No. 021496-002000US), Ser. No. 10/173,227 (Attorney Docket No. 021496-002300US), (now U.S. Pat. No. 6,790,173); Ser. No. 10/173,238 (Attorney Docket No. 021496-002400US), (now U.S. Pat. No. 6,837,847); and Ser. No. 10/173,220 (Attorney Docket No. 021496-002200US), (now U.S. Pat. No. 6,783,491), each of which was filed Jun. 13, 2002, and each of which is incorporated herein by reference in its entirety.

### BACKGROUND OF THE INVENTION

### FIELD OF THE INVENTION

The present invention relates to systems for endoluminal advancement through a hollow body organ. More particularly, the present invention relates to shapelockable disposable apparatus and methods for endoluminal advancement.

A physician performing a gastrointestinal examination or treatment commonly advances an endoscope through a patient's anus into the patient's colon. In order to permit full examination of the colon, the endoscope must be advanced up to the cecum. Advancement may be directed via a steerable distal end portion of the endoscope. However, at bends in the colon, e.g., at the sigmoid and especially at the two colonic flexures, advancement problems regularly occur, including a risk of injury, pain to the patient, cramp-like contractions of the colon, and even an inability to further advance the endoscope. Much of these problems occur because the colon is comprised of soft tissue which is weakly adhered to the abdomen.

The use of the endoscope for examining the interior of the intestinal tract is well-known. A complete examination typically requires the physician to advance the endoscope into the colon, negotiate the sigmoid colon, and left and right colic flexures up to the cecum. Advancement of the endoscope is generally accomplished by manipulation of a steerable tip of the endoscope, which is controlled at the proximal end of the device by the physician, in addition to torquing and pushing the scope forward or pulling it backward.

Other previously-known apparatus and methods use an overtube having variable rigidity, so that the overtube may be inserted through curved anatomy in a flexible state, and then selectively stiffened to resist bending forces generated by passing a colonoscope through the overtube.

While previously-known apparatus and methods provide some suggestions for solving the difficulties encountered in advancing diagnostic or therapeutic instruments through easily distensible body organs, few devices are commercially available. Moreover, other drawbacks of previously-known devices may be related to the complexity or cost of such devices or the lack of suitable materials.

In any event, there exists an un-met need for relatively inexpensive devices which not only provide a rigid platform for endoluminal advancement and for the insertion of diagnostic or therapeutic instruments in a hollow body organ, but which are also disposable, for instance, after a single use. Such a device is low-cost and easily manufacturable.

### BRIEF SUMMARY OF THE INVENTION

An example of a shapelock assembly may generally comprise an elongate body which defines at least one lumen therethrough for advancement of an endoscope or other endoscopic instruments therethrough. The handle assembly may be comprised generally of a handle body and locking handle which may be configured to actuate one or more cables routed throughout the elongate body such that a plurality of nested links comprising body are compressed against one another to transition the elongate body from a flexible state to a rigid shape-locked state.

Once in its shape-locked condition, the elongate body maintains any configuration in a rigid manner. Release of the locking handle relative to handle body releases the elongate body to transition back into a flexible body to conform into another configuration. An endoscope or any number of endoscopic instruments may be advanced into and through an entry lumen and elongate body to effect treatment. Further details and examples of shape-locking elongate bodies are disclosed in U.S. patent application Ser. No. 10/281,462 filed Oct. 25, 2002 (U.S. patent Pub. No. 2003/0233066 A1), which is incorporated herein by reference in its entirety.

When locked in a configuration, the elongate body of the shapelock assembly generally experiences compressive loads imparted upon the individual links in maintaining its shapelocked configuration. The links also experience loading forces from the manipulation and articulation of the endoscope through the assembly as well as from torquing and manipulation of the shapelock assembly itself by the physician. In particular, the links which are compressed against one another may deform, plastically or otherwise, particularly a lower portion of the link, i.e., the portion of the link about the inner surface, when compressed against an adjacent outer surface. Accordingly, the links are desirably configured and/or fabricated from materials having mechanical properties sufficient to withstand such forces and manipulation without failure.

One such material is a thermoplastic called Parmax®, which is a self-reinforced polymer having an inherent rigid-rod structure which does not require added fillers. Moreover, the cost of fabricating links from Parmax® allows for a lower cost of manufacturing the links relative to links made from other materials, such as titanium, stainless steel, aluminum, etc. Generally, Parmax® is a poly (paraphenylene) copolymer manufactured by Mississippi Polymer Technologies, Inc. in Bay St. Louis, Mo. and may be machined or molded to form the desired shape of link. Accordingly, the shapelock body may be fabricated from links made entirely from Parmax®.

Alternatively, one or more of the links may be fabricated from a composite link, i.e., a reinforced link. For instance, the reinforced link may be comprised of Parmax® or a thermoplastic having a reinforcing ring integrally formed as an outer ring of the link. The reinforcing ring may comprise any number of materials having sufficient strength, e.g., titanium, stainless steel, aluminum, nitinol, etc., to circumferentially buttress or reinforce the thermoplastic ring near or around areas of the links which may be particularly susceptible to deformation when under compressive loads. The reinforcing ring can be attached, integrated, or otherwise connected as an outer ring about an outer surface of link, an inner ring about an inner surface of the link, or as a lower reinforcing ring replacing the entire lower portion of link.

In further variations, the entire link or portions of the link may be covered or coated with another material to enhance the strength of the link. Accordingly, a reinforcing layer or coating may be deposited over a surface of the link.

In others variations for the shapelock body, a partial hybrid linked body may be utilized in which thermoplastic or Parmax(® links are used in combination with reinforced or metallic links in an alternating configuration. Links fabricated from thermoplastic or Parmax® may be interspersed with links fabricated from metals or metallic alloys such as titanium, aluminum, etc. Alternatively, the links may be interspersed with metallic inserts comprised of a stamped or molded metallic sleeve or covering which may be placed between adjacent links.

In yet another variation, the shapelock body may be formed of reinforced links along a first section of the body and of links fabricated from a thermoplastic or Parmax® along a second section. Moreover, the shapelock body may be divided into more than two sections, e.g., three or more, in which each section may be comprised of any combination of links described herein.

### BRIEF DESCRIPTION OF THE DRAWINGS

FIG. 1 illustrates a shapelock assembly defining at least one lumen therethrough.

FIG. 2 illustrates an assembly view of an exposed elongate shapelocking body and a liner assembly which may be disposed upon and within the elongate body.

FIGS. 3A to 3C illustrate an example of one method for inserting a shapelock assembly into a patient body.

FIG. 4 illustrates an alternative method for inserting both an endoscope and shapelock assembly into the patient body.

FIGS. 5A and 5B show cross-sectional and exploded assembly views of a portion of the shapelock body, respectively, illustrating the relative positioning of adjacent links.

FIGS. 6A and 6B show top and perspective views, respectively, of a link from the shapelock body having a reinforcing ring integrated with the link.

FIG. 7A shows a partial cross-sectional perspective view of a link with a reinforcing ring integrated therewith over the outer diameter of the link.

FIG. 7B shows a perspective view of the reinforcing ring from FIG. 7A.

FIG. 8A shows a partial cross-sectional perspective view of a link with a reinforcing ring integrated therewith over the inner diameter of the link.

FIG. 8B shows a perspective view of the reinforcing ring from FIG. 8A.

FIG. 9A shows a partial cross-sectional perspective view of a link with a reinforcing ring integrated therewith replacing an entire lower portion of the link.

FIG. 9B shows a perspective view of the reinforcing ring from FIG. 9A.

FIG. 10A and 10B illustrate partial cross-sectional profiles of various reinforced links having a reinforcing layer or coating deposited over an entire or partial outer surface of the link, respectively.

FIG. 11A shows a perspective view of an alternative reinforcing ring having one or more projections for secure attachment to the link.

FIGS. 11B to 11H show examples of alternative variations for the projections which may be utilized on a reinforcing ring.

FIG. 12 shows a cross-sectional view of a partial hybrid linked body in which thermoplastic or Parmax® links may be used in combination with reinforced or metallic links in an alternating configuration.

FIG. 13 shows another variation of a hybrid linked body which may be comprised of links interspersed with metallic inserts.

FIG. 14 illustrates a shapelock body which may be comprised of different types of links along multiple sections of the shapelock body, e.g., reinforced links along a first section and links fabricated from a thermoplastic or Parmax® along a second section.

### DETAILED DESCRIPTION OF THE INVENTION

Generally in use, an endoscope may be advanced into a patient's body lumen, such as the lower gastro-intestinal tract via the anus or the upper gastro-intestinal tract via the patient's mouth. However, the tissue of the colon and small intestines are typically unsupported and advancement through these body lumens is difficult. Furthermore, looping of the tissue and unraveling of pleated tissue relative to the endoscope makes endoscopic advancement particularly difficult. Accordingly, providing a stable platform through which the endoscope may be endoluminally advanced may facilitate the endoluminal manipulation of the endoscope and examination of the tissue.

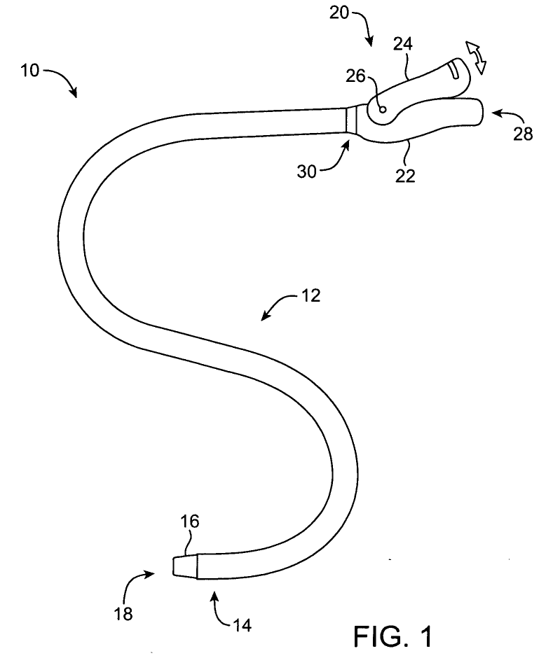

An example of a stable endoluminal platform device is shown in shapelock assembly 10 in FIG. 1. Shapelock assembly 10 may generally comprise an elongate body 12 which defines at least one lumen 18 therethrough for advancement of an endoscope or other endoscopic instruments therethrough. A distal tip 16, which may be configured into an atraumatic shape, may be positioned near or at the distal end 14 of elongate body 12. Handle assembly 20 may be coupled to a proximal end of elongate body 12.

Handle assembly 20 may be comprised generally of handle body 22 and locking handle 24 which may be configured to actuate one or more cables routed throughout elongate body 12 such that a plurality of nested links, in part comprising body 12 and as described below in further detail, are compressed against one another to transition elongate body 12 from a flexible state to a rigid shape-locked state. Once in its shape-locked condition, elongate body 12 maintains any configuration in a rigid manner. Release of locking handle 24 relative to handle body 22 releases elongate body 12 to transition back into a flexible body to conform into another configuration.

Locking handle 24 may be rotatably coupled to handle body 22 via pivot 26 such that rotation of locking handle 24 in the direction shown in FIG. 1 against handle body 22 may actuate the shape-locking feature of elongate body 12. However, any number of actuation mechanisms as generally known may also be utilized. Handle body 22 may also define in its proximal end an entry lumen 28 which extends through handle assembly 20 and elongate body 12. The proximal end of elongate body 12 may be coupled or otherwise attached to handle assembly 20 at handle interface 30. As mentioned above, an endoscope or any number of endoscopic instruments may be advanced into and through entry lumen 28 and elongate body 12 to effect treatment through assembly 10. Further details and examples of shape-locking elongate bodies are disclosed in U.S. patent application Ser. No. 10/281,462 filed Oct. 25, 2002 (U.S. patent Pub. No. 2003/0233066 A1), which is incorporated herein by reference in its entirety.

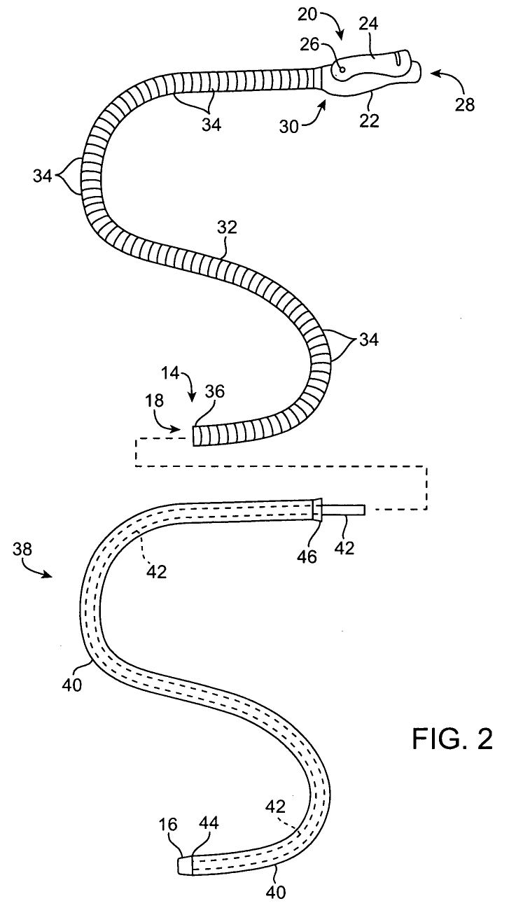

As mentioned above and as shown in FIG. 2, the shape-locking elongate body 12 is generally comprised of an underlying body 32 having a plurality of nested links 34 which are slidable relative to one another. Each link 34 may define one or more openings therethrough such that the stacked links 34 collectively form lumen 18 through the length of the device. The terminal link 36 positioned near or at the distal end of the link body 32 may anchor one or several control wires which are routed through the length of body 32. Overlying the linked body 32 is a liner or covering assembly 38. An inner liner or layer 42 may typically comprises a soft elastomeric and/or hydrophilic coated material, such as silicon or synthetic rubber, and extends through lumen 18 of nestable links 34 to a liner for the lumen 18. Inner liner 42 may extend from distal tip 16 and proximally through handle assembly 20 to terminate externally of or at entry lumen 28.

An outer liner 40, which may be formed into a flexible elastomeric covering, may also extend from distal tip 16 over inner liner 42 such that outer and inner liners 40, 42 may be integrally formed with one another in attachment 44 at distal tip 16. When inner liner 42 is positioned within lumen 18 and outer liner 40 is disposed over body 32 to encapsulate the links 34, the proximal end of outer liner 40 may be connected or otherwise attached, e.g., via a temporary mechanical connection, via handle locking interface 46 at the proximal end of outer liner 40 to handle interface 30. Outer liner 40, when disposed over links 34, provides a relatively smooth outer surface for elongate body 12 and aids in preventing tissue from being captured or pinched during relative rotation of adjacent nestable links 34. Further examples and descriptions of the liner assembly 38 and its positioning upon the shapelocking assembly 10 maybe seen in further detail in U.S. patent application Ser. No. 11/115,947 filed Apr. 26, 2005, which is incorporated herein by reference in its entirety.

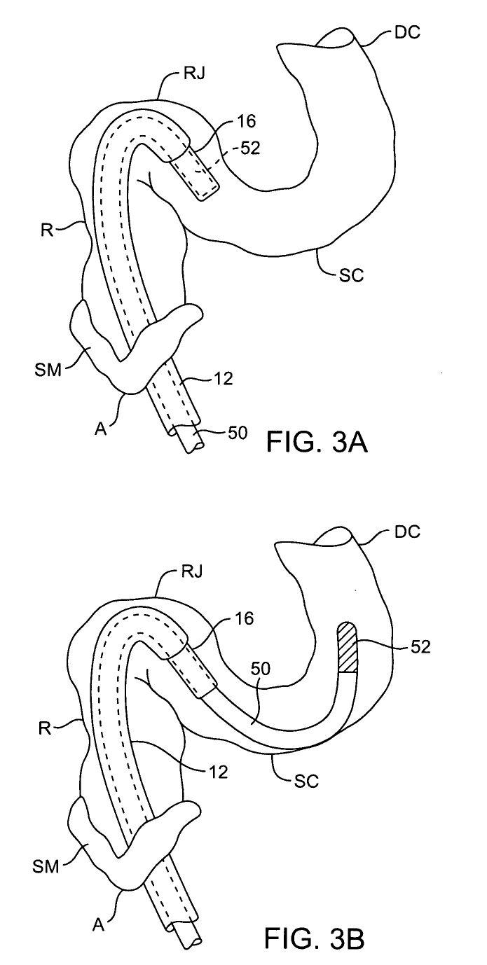

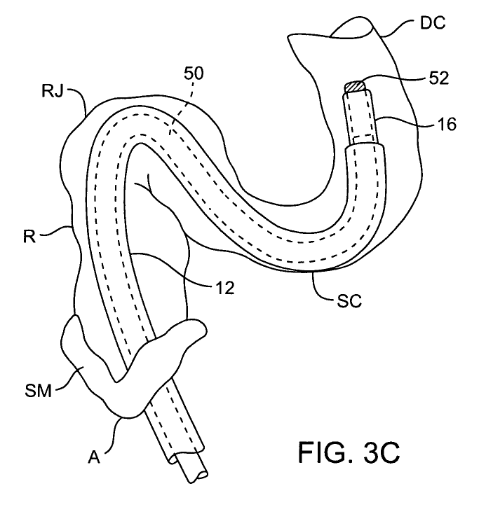

Referring to FIGS. 3A to 3C, an example of one method of utilizing shapelock assembly 50 is described. Endoscope 50 and elongate body 12 may be inserted into the patient either simultaneously or by first back-loading the elongate body 12 onto the endoscope 50. To perform simultaneous insertion, endoscope 50 may be introduced into entry lumen 28 of handle assembly 20 until the steerable distal tip 52 of the endoscope 50 is disposed in the distal end 14 of shapelock assembly 10. As one unit, endoscope 50 and elongate body 12 are inserted, e.g., into rectum R of the patient, and navigated about rectosigmoid junction RJ, as shown in FIG. 3A.

Once distal tip 52 and distal tip 16 (if utilized) have been negotiated past rectosigmoid junction RJ, the current shape of elongate body 12 may be shape-locked in the manner discussed above to provide a rigid channel through which endoscope 50 may be further advanced into the colon without distending rectosiginoid junction RJ, as shown in FIG. 3B. Once distal tip 52 of endoscope 50 is negotiated past sigmoid colon SC, elongate body 12 may be released from its rigid state and advanced along endoscope 50 until it too traverses sigmoid colon SC, as shown in FIG. 3C. Again, the current shape of elongate body 12 may be locked to provide a rigid channel for advancement of endoscope 50. To negotiate the remainder of the colon, such as left colic flexure LCF and right colic flexure RCF, the preceding steps may be repeated. In this manner, endoscope 50 and elongate body 12 may be navigated through the tortuous curves of the colon without distending the colon, and thereby causing discomfort, spasm or injury.

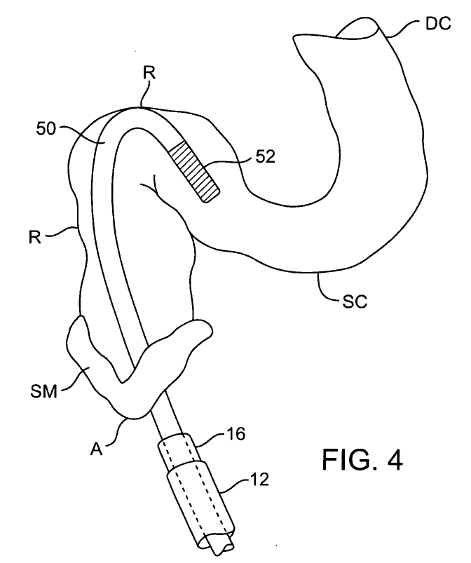

Alternatively, rather than simultaneously inserting both endoscope 50 and elongate body 12 into the patient, shapelock assembly 10 first may be back-loaded onto the endoscope 50. Elongate body 12 may be threaded onto endoscope 50 and positioned proximally of endoscope steerable distal tip 52, as shown in FIG. 4. Endoscope 50 may then be inserted into rectum R of the patient and advanced around rectosigmoid junction RJ. Elongate body 12 may then be advanced along endoscope 50 into rectum R of the patient, using endoscope 50 as a guide to negotiate rectosigmoid junction RJ. Once elongate body 12 traverses rectosigmoid junction RJ to the position shown in FIG. 3A, the shape of elongate body 12 may be locked to provide a rigid channel through which endoscope 50 may be further advanced into the colon. To negotiate the remainder of the colon, the steps discussed with reference to FIGS. 3B and 3C may be performed.

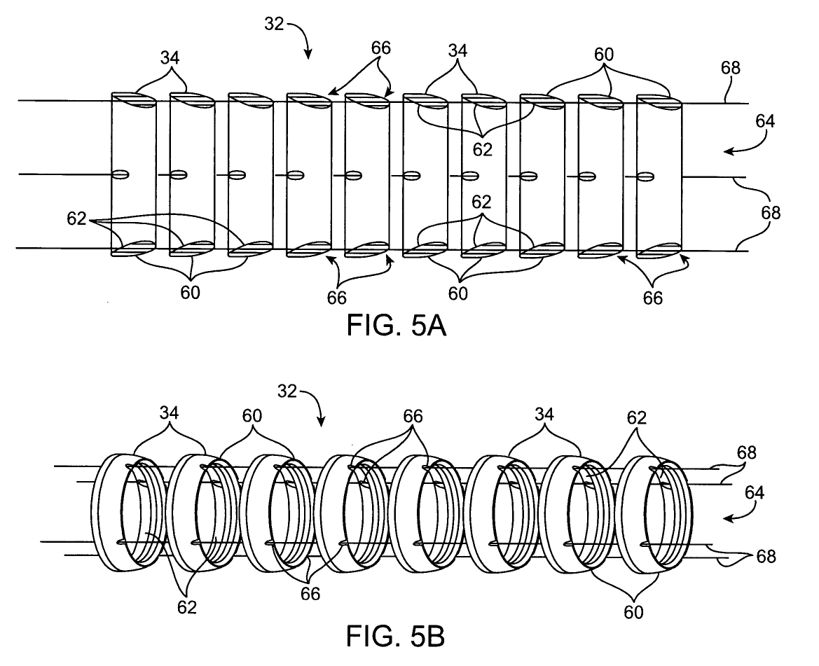

FIGS. 5A and 5B show cross-sectional and exploded assembly views of a portion of shapelock body 32, respectively, illustrating the relative positioning of adjacent links. For purposes of illustration in both FIGS. 5A and 5B, nestable links 34 are shown spaced-apart, but it should be understood that links 34 are disposed so that their adjacent outer surfaces 60 and inner surfaces 62 coact with one another. Each of nestable links 34 has a central lumen 64 to accommodate endoscope 50, as described above, and preferably three or more tension wire lumens 66. When assembled as shown above, nestable links 34 may be fastened such that adjacent surfaces 60 and 62 are disposed in a coacting fashion by a plurality of tension wires 68 that extend through respective tension wire lumens 66.

Adjacent surfaces 60 and 62 of each nestable link 34 are contoured to mate with the next adjacent link, so that when tension wires 68 are relaxed, surfaces 60 and 62 can rotate relative to one another. The distal ends of tension wires 68 may be fixedly connected to the distal end of shapelock assembly 10, as mentioned above, and the proximal ends of tension wires 68 may be fixedly connected to a tensioning mechanism disposed within handle assembly 20. When actuated by locking handle 24, tension wires 68 impose a load that clamps adjacent surfaces 60 and 62 of nestable links 34 together at the current relative orientation, thereby fixing the shape of shapelock assembly 10.

When the load in tension wires 68 is released, tension wires 68 provide for relative angular movement between nestable links 34. This in turn renders shapelock assembly 10 sufficiently flexible to negotiate a tortuous path through the body. When the tensioning mechanism is actuated, however, tension wires 68 are retracted proximally to apply a clamping load to the nestable links. This load prevents further relative movement between adjacent links 34 and stiffens shapelock assembly 10 so that any distally directed force applied to endoscope 50 causes distal steerable tip 52 to advance further into the colon, rather than causing shapelock assembly 10 to bear against the wall of the colon. The shapelock assembly 10 absorbs and distributes vector forces, shielding the tissue wall.

With respect to the individual nestable links 34, these links have been previously described in U.S. patent application Ser. No. 10/281,462 as being fabricated from any number of polymers filled with fibers of glass, carbon, or combinations thereof. For example, links 34 may be molded from polyurethane filled with 20-40% by volume of glass fibers, 20-40% by volume of carbon fibers, or 20-40% by volume of glass and carbon fibers. Alternatively or additionally, the links may also be molded or machined from other polymers and/or metals, such as polyurethane, polyvinyl chloride, polycarbonate, nylon, titanium, tungsten, stainless steel, aluminum, etc., or combinations thereof.

When locked in a configuration, the elongate body 12 of shapelock assembly 10 generally experiences compressive loads imparted upon the individual links 34 in maintaining its shapelocked configuration. The links 34 also experience additional loading forces from the manipulation and articulation of the endoscope 50 through the assembly 10 as well as from torquing and manipulation of the shapelock assembly 10 itself by the physician. In particular, links 34 which are compressed against one another may deform, plastically or otherwise, a lower portion of the link 34, i.e., the portion of the link about inner surface 62, when compressed against an adjacent outer surface 60. Accordingly, the links 34 are desirably configured and/or fabricated from materials having mechanical properties sufficient to withstand such forces and manipulation without failure.

One such material which may be particularly suited for use in fabricating the links 34 is a thermoplastic called Parmax®, which is a self-reinforced polymer having an inherent rigid-rod structure which does not require added fillers. Moreover, the cost of fabricating links 34 from Parmax® allows for a lower cost of manufacturing the links 34 relative to links 34 made from other materials, such as titanium, stainless steel, aluminum, etc. Generally, Parmax® is a poly (paraphenylene) copolymer manufactured by Mississippi Polymer Technologies, Inc. in Bay St. Louis, Mo. and may be machined or molded to form the desired shape of link 34. Such a material may provide sufficient strength to withstand the compressive and dynamic forces imparted upon the links 34. Accordingly, the shapelock body 32 shown in FIGS. 5A and 5B may be fabricated from links 34 made entirely from Parmax®.

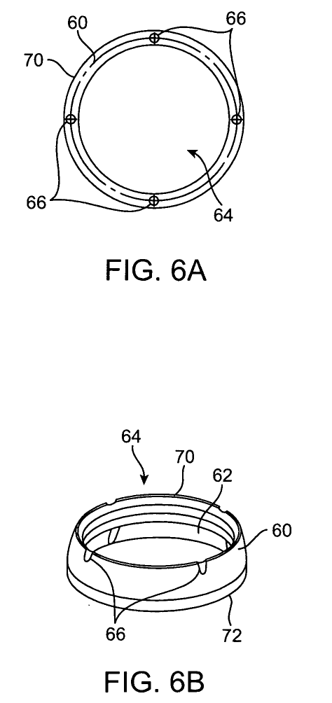

One or more of the links 34 in the shapelock body 32 may be fabricated alternatively from a composite link. As shown in the top and perspective views of FIGS. 6A and 6B, respectively, one or more of the links of shapelock body 32 may be a reinforced link 70. For instance, reinforced link 70 may be comprised of Parmax® or a thermoplastic having a reinforcing ring 72 integrally formed as an outer ring of the link 70. Reinforcing ring 72 may comprise any number of materials having sufficient strength, e.g., titanium, stainless steel, aluminum, nitinol, etc., to circumferentially buttress or reinforce the thermoplastic ring 70 near or around areas of the links which may be particularly susceptible to deformation when under compressive loads. FIG. 6B shows reinforcing ring 72 attached, integrated, or otherwise connected as an outer ring 72 about an outer surface of link 70 below outer surface 60. If reinforcing ring 72 is integrated as an outer ring, the ring desirably presents a smooth transitional surface between the ring 72 and the outer surface of the link 70 so as to minimize any physical discontinuities between the two.

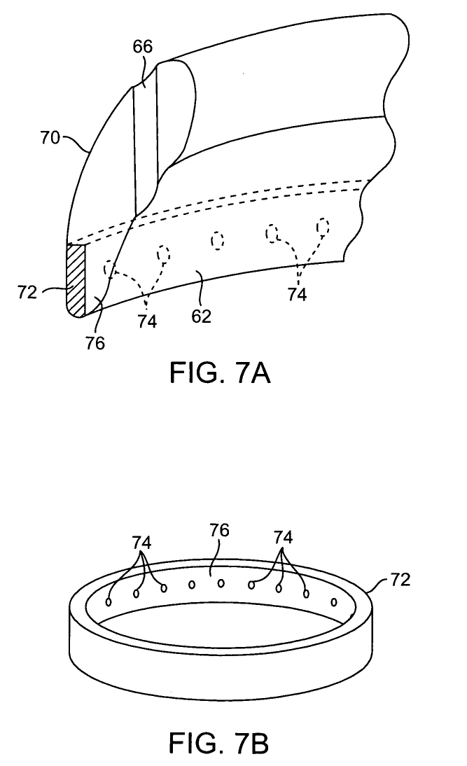

FIG. 7A shows a partial cross-sectional perspective view of link 70 with its reinforcing ring 72 integrated therewith over the outer diameter of link 70. To facilitate the attachment or connection of reinforcing ring 72 to a ring contact surface 76 along link 70, one or more openings or bores 74 may be defined along ring inner surface 76, as shown in the perspective view of reinforcing ring 74 in FIG. 7B. These one or more openings 74 may be spaced uniformly around inner surface 76 of ring 72 to provide areas within which the Parmax® or thermoplastic material may flow into at least partially so as to provide a mechanical bond or attachment between ring 72 and link 70. Although openings 74 are shown as uniformly-spaced features, alternative configurations such as grooves or slots may also be utilized.

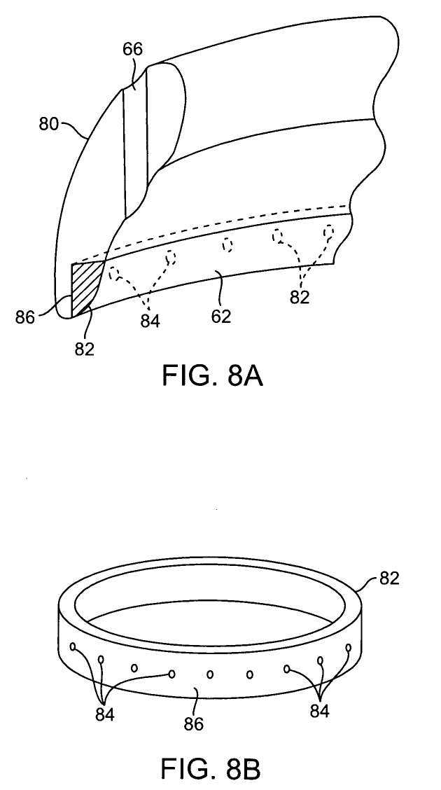

An alternative composite link 80 may be seen in the partial cross-sectional perspective views of link 80 and inner ring 82 in FIGS. 8A and 8B, respectively. Composite link 80 may be molded or machined and assembled similarly to link 70 described above but with inner ring 82 formed or adhered to the inner surface 62 of link 80. Inner ring 82 may also have one or more openings or bores 84 defined over its outer surface 86, as shown in FIG. 8B, for facilitating the attachment between inner ring 82 and link 80. Moreover, rather than utilizing openings 84 for receiving flow of the link material within, adhesive, cement, epoxy, etc., may alternatively be utilized for attaching the two portions not only in this variation, but other variations of the links described herein.

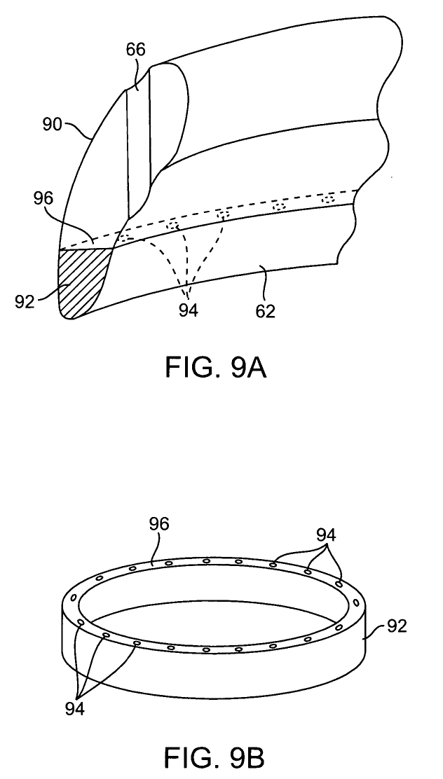

In yet another variation of a composite link, FIGS. 9A and 9B show partial cross-sectional perspective views of link 90 and lower reinforcing ring 92 in FIGS. 9A and 9B, respectively. In this variation, reinforcing ring 92 may replace the entire lower portion of link 90, as shown. As above, reinforcing ring 92 may be attached or otherwise connected to link 90 via one or more openings or bores 94 defined over an upper surface 96 of ring 92, as shown in FIG. 9B. Because ring 92 replaces the entire lower portion of link 90 in this variation, ring 92 approximates the profile or shape of the lower portion or link 90.

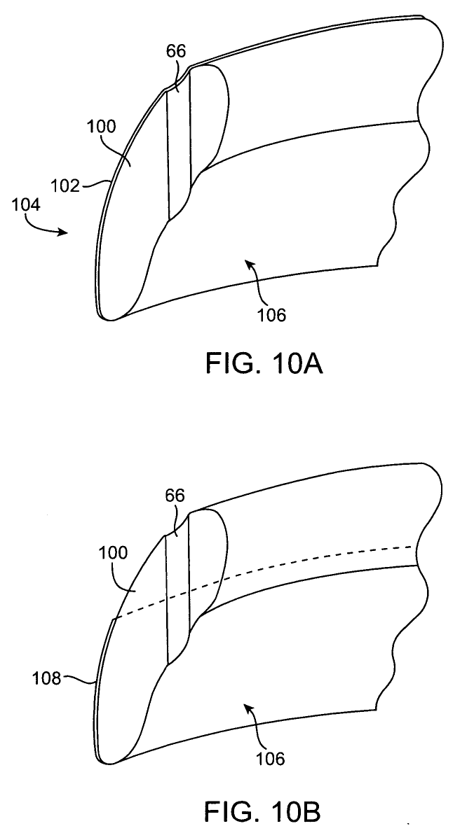

In further variations, rather than replacing or reinforcing portions of the link with reinforcing rings, the entire link or portions of the link may be covered or coated with another material to enhance the strength of the link. As shown in the partial cross-sectional profile of reinforced link 100 of FIG. 10A, a reinforcing layer or coating 102 may be deposited over a surface of the link 100. Although FIG. 10A shows layer or coating 102 deposited upon an outer surface 104 of the link 100, coating 102 may alternatively be deposited over the entire inner 106 and outer 104 surfaces of link 100. Hard thin-film coatings may be deposited upon the link surfaces utilizing various procedures such as physical vapor deposition (PVD) or chemical vapor deposition (CVD). Moreover, various materials such as ceramics, metals and metallic alloys such as chromium, aluminum, titanium, nickel, etc., as well as composites utilizing diamond coatings, silicon carbide, etc., may be utilized for the coating materials.

As mentioned, the reinforcing layer or coating may be deposited partially over the surface of link 100. As shown in FIG. 10B, reinforcing layer 108 may be deposited partially over the lower outer surface of link 100. Moreover, other coating configurations may also be utilized on a single link, a plurality of links, or just a few of the links in shapelock body 32.

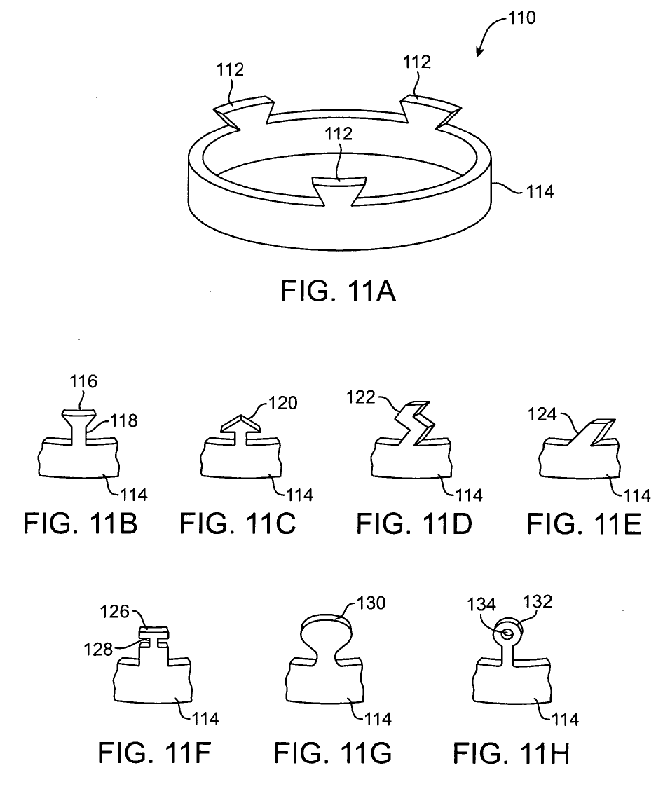

In the case of a reinforcing ring attached or connected to a thermoplastic link, as 30 described above, various alternative configurations may be adopted for the ring shape to ensure a secure connection between the two. As shown in FIG. 11A, reinforcing ring 110 may be utilized, e.g., in place of ring 92 above. Ring 110, in this variation, may have a ring body 114 with one or more projections 112 extending from the ring body 114. These projections 112, shown in this variation as an inverted partial triangular shape, are configured to securely fit into a complementary pattern defined in the link and are generally shaped to resist or inhibit detachment of the ring body 114 from the link.

Other examples of such mechanical securing projections are shown in FIGS. 11B to 11H. Although these examples illustrate specific configurations, these are intended merely to be illustrative and are not limited to the various configurations shown. Other shapes which inhibit or resist ring detachment from the link may also be utilized. Moreover, these and other shapes may be utilized in different combinations with various configurations on individual links, as so desired. FIG. 11B shows an inverted triangular shape 116 extending from a post 118. FIG. 11C shows a triangular shape also extending from a post. FIG. 11D shows an angled projection 122 having multiple angles while FIG. 11E shows a single angled projection 124. FIG. 11F shows a variation having a protrusion 126 delineated by a notch-out 128. FIG. 11G shows a variation of a circularly-shaped protrusion 130 while FIG. 11H shows a variation of a circularly-shaped protrusion 132 having an eyelet 134 defined therethrough within which link material may be flowed.

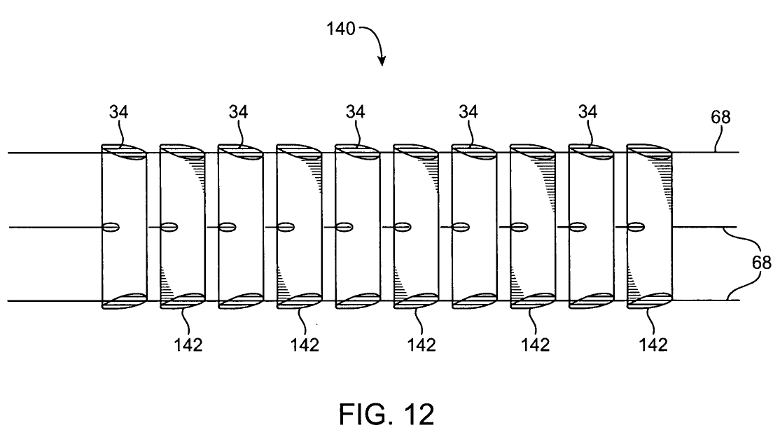

In others variations for the shapelock body, various alternatives may be utilized. For example, FIG. 12 shows a cross-sectional view of a partial hybrid linked body 140 in which thermoplastic or Parmax® links may be used in combination with reinforced or metallic links in an alternating configuration. As shown, links 34 fabricated from thermoplastic or Parmax® may be interspersed with links 142 fabricated from metals or metallic alloys such as titanium, aluminum, etc. Alternatively, link 142 may comprise any of the reinforced links described above.

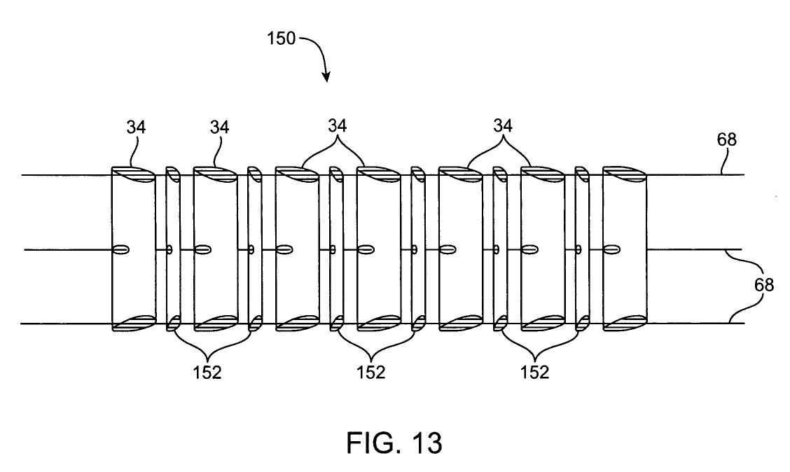

Alternatively, hybrid linked body 150 may be comprised of links 34 interspersed with metallic inserts 152, as shown in FIG. 13. Metallic inserts 152 may simply comprise a stamped or molded metallic sleeve or covering which may be placed between adjacent links 34.

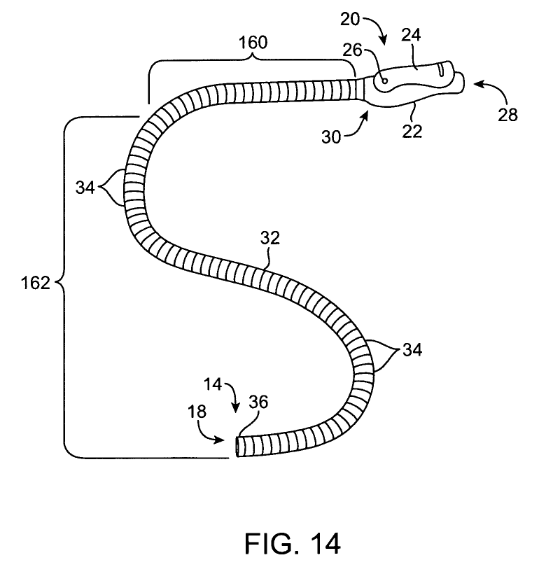

In yet another variation, shapelock body 32 may be formed of reinforced links along a first section 160 of body 32 and of links 34 fabricated from a thermoplastic or Parmax® along a second section 162, as shown in FIG. 14. Alternatively, the links along first section 160 may be fabricated from metallic links while the links along second section 162 may comprise thermoplastic or Parmax® links or reinforced links. Moreover, shapelock body 32 may be divided into more than two sections, e.g., three or more, in which each section may be comprised of any combination of links described herein.

Although various illustrative variations are described above, it will be evident to one skilled in the art that a variety of combinations of aspects of different variations, changes, and modifications are within the scope of the invention. It is intended in the appended claims to cover all such combinations, changes, and modifications.

What is claimed is:

1. A system for advancing through a hollow body organ, comprising:
an elongate body adapted to transition between a flexible state and a rigidized state, wherein the elongate body defines at least one lumen therethrough and is comprised of a plurality of nested links made from poly (paraphenylene) copolymer.

2. The system of claim 1 further comprising a handle assembly coupled to a proximal end of the elongate body and adapted to actuate the elongate body between the flexible state and the rigidized state.

3. The system of claim 1 further comprising a liner assembly having an inner liner for insertion through the at least one lumen and an outer liner for placement over the elongate body, wherein a distal end of the inner liner and a distal end of the outer liner are fixedly attached.

4. The system of claim 1 wherein the poly (paraphenylene) copolymer comprises a self-reinforced polymer having an inherent rigid-rod structure.

5. The system of claim 1 wherein at least one of the nested links further comprises a reinforcing ring integrally formed with the link and configured to circumferentially buttress or reinforce the link.

6. The system of claim 5 wherein the reinforcing ring is integrated along an outer surface of the at least one nested link.

7. The system of claim 5 wherein the reinforcing ring is integrated along an inner surface of the at least one nested link.

8. The system of claim 5 wherein the reinforcing ring is integrated along a lower portion of the at least one nested link.

9. The system of claim 5 wherein the reinforcing ring is made from titanium, stainless steel, aluminum, or nitinol.

10. The system of claim 5 wherein the reinforcing ring and the at least one nested link presents a smooth transitional surface.

11. The system of claim 5 wherein the reinforcing ring defines a plurality of openings or bores along a surface for contacting the at least one nested link.

12. The system of claim 5 wherein the reinforcing ring comprises at least one projection for connection to the at least one nested link, the projection being configured to inhibit detachment between the link and reinforcing ring.

13. The system of claim 5 wherein the elongate body comprises alternating nested links having the reinforcing ring.

14. The system of claim 5 wherein the elongate body comprises at least a first section comprising a plurality of nested links each having a reinforcing ring and at least a second section distal of the first section comprising a plurality of nested links.

15. The system of claim 1 wherein at least one of the nested links is at least partially covered or coated to enhance a strength of the link.

16. The system of claim 15 wherein the at least one nested link is covered or coated via physical vapor deposition or chemical vapor deposition.

17. The system of claim 1 wherein the plurality of nested links comprises a first link made from poly (paraphenylene) copolymer and a second link having a reinforcing ring integrally formed with the link such that the first link and the second link are positioned in an alternating manner.

18. A method for advancing a diagnostic or therapeutic instrument into an unsupported, hollow body organ, comprising:
providing an elongate body adapted to transition between a flexible state and a rigidized state, wherein the elongate body defines at least one lumen therethrough and is comprised of a plurality of nested links made from poly (paraphenylene) copolymer;
inserting the elongate body and the diagnostic or therapeutic instrument into the unsupported, hollow body organ;
rigidizing the elongate body while disposed within the unsupported, hollow body organ;
transitioning the elongate body into its flexible state; and
withdrawing the elongate body and the diagnostic or therapeutic instrument from the unsupported, hollow body organ.

19. The method of claim 18 further comprising disposing the elongate body.

20. The method of claim 1 wherein providing comprises providing at least one nested link having a reinforcing ring integrally formed with the link and configured to circumferentially buttress or reinforce the link.

21. The method of claim 20 further comprising providing an elongate body having alternating nesting links having the reinforcing ring.

22. The method of claim 20 further comprising providing an elongate body having at least a first section comprising a plurality of nested links each having a reinforcing ring and at least a second section distal of the first section comprising a plurality of nested links.
